(19) United States
(12) Patent Application Publication
(10) Pub. No.: US 2022/0223235 A1
(43) Pub. Date: Jul. 14, 2022

(54) SPECTRAL CLASSIFICATION SYSTEMS AND METHODS

(71) Applicant: FLIR Detection, Inc., Stillwater, OK (US)

(72) Inventors: Douglas K. Martins, Lafayette, IN (US); Dennis Barket, JR., West Lafayette, IN (US)

(21) Appl. No.: 17/567,068

(22) Filed: Dec. 31, 2021

Related U.S. Application Data
(60) Provisional application No. 63/137,094, filed on Jan. 13, 2021.

(51) Int. Cl. G16C 20/70; G16C 10/00

(52) U.S. Cl. CPC G16C 20/70; G16C 10/00

(57) ABSTRACT

Various techniques are provided for training a neural network to classify chemical spectra data, such as Ion Mobility Spectrometry data. Machine learning models are trained using a training dataset comprising labeled chemical spectra data. The training process tracks chemical classification-related metrics and other informative metrics as the training dataset is processed. The trained models are tested using a validation dataset of chemical spectra data to generate performance results. A model analysis engine extracts and analyzes the informative metrics and performance results, generates parameters for a modified training dataset and features to improve model performance, and generates corresponding instructions to generate a new training dataset. The process repeats in an iterative fashion to build a final training dataset and set of models of classifying one or more chemicals.

### CROSS-REFERENCE TO RELATED APPLICATIONS

This application claims priority to and the benefit of U.S. Provisional Patent Application No. 63/137,094 filed Jan. 13, 2021 and entitled “SPECTRAL CLASSIFICATION SYSTEMS AND METHODS,” which is incorporated herein by reference in its entirety.

### TECHNICAL FIELD

Embodiments of the present disclosure relate generally to chemical detection systems and methods and, more particularly for example, to systems and methods for classification and/or analysis of chemical sensor data in mobile devices.

### BACKGROUND

Field-deployable chemical sensing devices are often limited by size, weight, power and cost (SWaP-C) constraints. For example, a chemical sensor may be configured to couple gas chromatography with mass spectrometry (GC-MS) for chemical identification and quantification of complex vapor mixtures. A vacuum system is often required for mass spectrometers, which drives much of the SWAP-C requirements of GC-MS systems. Ion Mobility Spectrometry (IMS) operated at atmospheric pressure provides a cheaper alternative to GC-MS, but conventional systems come with a trade-off of increased false alarms and limited specificity. Advancements in IMS technology such as Differential Mobility Spectrometry (DMS, also known as high-Field Asymmetric Ion Mobility Spectrometry, (FAIMS)) utilize smaller ion separation regions, higher electric fields and electric field manipulation to take advantage of the dependence of ion mobility and thermal decomposition on electric field strength. While these advances have improved the specificity of IMS, classification of target responses remain highly empirical and subject to environmental conditions.

In view of the foregoing, there is a continued need for improved chemical sensor systems and methods, including IMS systems that are field-deployable for use in chemical detection, classification and/or quantification of complex vapor mixtures.

### SUMMARY

Systems and methods are provided for improved spectral classification and analysis of chemical spectra. Chemical classification systems and methods, including systems and methods for training, validating and selecting models for chemical classification are disclosed herein. In one or more embodiments, a training dataset is defined and chemical features (such as analyte features) are generated and used to train a plurality of models (such as a convolutional neural network (CNN)) for chemical detection and classification. A chemical classification training dataset is generated to train one or more models, the training results are validated for each model using a separate validation dataset, and a model analysis engine analyzes informative metrics and performance results to modify the datasets, features, models, parameters and other data to optimize the models during a next iteration.

The scope of the invention is defined by the claims, which are incorporated into this section by reference. A more complete understanding of embodiments of the invention will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

FIG. 1A illustrates a chemical classification training and validation system, in accordance with various embodiments of the present disclosure.

FIG. 1B is a flow diagram illustrating an example operation of the chemical classification training and validation system of FIG. 1A, in accordance with various embodiments of the present disclosure.

FIG. 1C is a chart illustrating example IMS spectra, in accordance with various embodiments of the present disclosure.

FIG. 2 illustrates an example chemical classification training and validation system, in accordance with various embodiments of the present disclosure.

FIG. 3 illustrates an example chemical classification mobile system, in accordance with various embodiments of the present disclosure.

FIG. 4A illustrates an example neural network training process for classifying chemicals, in accordance with various embodiments of the present disclosure.

FIG. 4B illustrates a model validation process for the neural network of FIG. 4A, in accordance with various embodiments of the present disclosure.

FIG. 5 illustrates an example process for generating training data and chemical classification models, in accordance with various embodiments of the present disclosure.

FIG. 6 illustrates a chemical classification system, in accordance with various embodiments of the present disclosure.

Embodiments of the disclosure and their advantages are best understood by referring to the detailed description that follows. It should be appreciated that like reference numerals are used to identify like elements illustrated in one or more of the figures.

### DETAILED DESCRIPTION

Ion mobility spectrometry (IMS), and its evolutions such as high-Field Asymmetric Waveform Ion Mobility Spectrometry (FAIMS) and Rapid Thermal Modulation Ion Spectrometry (RTMIS), produce data that is more complex than many technologies deployed in mobile chemical sensing systems, such as mass spectrometry. Unlike mass spectrometry data which is typically represented as a graph with a single independent variable, IMS, FAIMS and RTMIS responses to chemical compounds are non-linear with respect to environmental conditions and system configuration. Thus, it is often more challenging, time-consuming and often highly empirical to develop chemical detection and classification systems that can reliably classify chemical compounds from IMS spectra data in real-world implementations.

In the present disclosure, novel machine learning approaches are used to classify compounds based on the unique chemical spectrum produced by a FAIMS, RTMIS and/or similar chemical sensing technology to decrease the development time, decrease the amount of data that is needed to develop classifiers, increase the efficiency of the data collection process, reduce the number of false positives generated by trained models, and/or provide more flexibility to incorporate emerging chemical libraries into trained models.

In various embodiments of the present disclosure, the use of statistical classification and machine learning algorithms for two-dimensional spectra (e.g., IMS spectra data) is used to provide a fast, flexible and accurate alternative to empirical development of classifiers for chemical sensors. Prediction models based on statistical algorithms can identify differences in spectral responses between varying analytes, ultimately increasing the specificity of the analytical instrumentation. The algorithms disclosed herein may include and support, but are not limited to, Decision Trees, Support Vector Machines, Logistic Regression, KNeighbors, Naïve Bayes, Ensemble methods (e.g. Random Forests), and Neural Networks.

This disclosure further describes a set of software tools and methods that are used to process chemical data (e.g., IMS spectra data), define unique features to differentiate the detection instrument response based on the chemical target, and develop a predictive model to classify an instrument response in the field. To develop these classification models, a dataset of chemical spectra that represents targets of interest, mixtures, interferents and environmental sampling conditions is collected and used to train a plurality of models. In some embodiments, the dataset includes rows of observations with each row containing features that include unique characteristics that differentiate one analyte from other analytes in the dataset. Examples of features may include peak height and peak location in the 2-D IMS spectrum. Other features may be identified by determining which features have the most value for classification, such as be using supervised and/or unsupervised statistical methods.

After features are identified, a dataset is created and split into training and validation subsets. The training dataset will be used to develop one or more classification models, while the validation dataset will be used to evaluate the model for accuracy. In some embodiments, a variety of classification models may be evaluated, optimized and compared to determine model(s) that provide the best performance for a desired application. The present disclosure describes methods for multiple models to be trained and validated on the same datasets, so that direct comparisons can be made. These methods allow the incorporation of new data, features and model parameters to iteratively tune the models and expand chemical libraries.

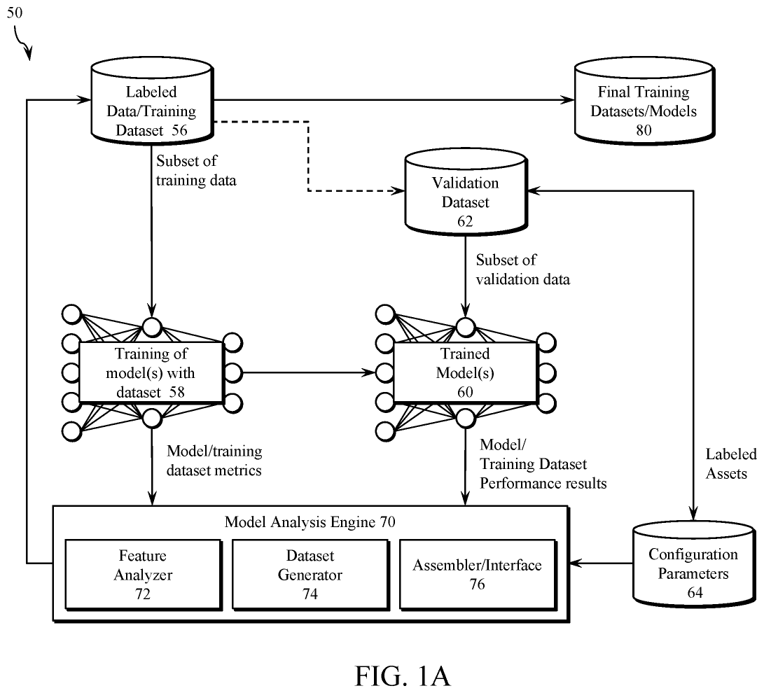

Referring to FIG. 1A, various embodiments of a system for training, validating, evaluating and selecting one or more models for classifying one or more chemicals will be described. A system 50 generates a training dataset and validated models in an iterative process that yields high performance models for chemical classification. The trained models are optimized to classify one or more analytes and the system 50 provides flexibility to incorporate additional target analytes as needed for particular implementations. In the illustrated embodiment, the system 50 includes a model analysis engine 70 configured to operate with a training dataset 56 (e.g., a set of labeled chemical data) for training different machine learning models (e.g., a neural network) in a training process 58. The training process 58 applies one or more subsets of training data from the training dataset 56 to produce trained classification models 60 and also generates model and/or training dataset metrics during the training process. Each of the trained classification models 60 is validated using a validation dataset 62, which may be selected as a subset of the training data that is not used for training the model.

In various embodiments, the training dataset 56 includes a plurality of labeled chemical data samples, and the validation dataset is a subset of the training dataset 56 that has not been used for training the models. The validation dataset 62 is input to the trained models 60 to classify each sample and the output classification is compared against the corresponding label to measure of the performance of a trained model 60. The training datasets 56 may include a variety of chemical samples representing a range of real world use cases for training and validating the models. The real-world data samples may be captured, for example, using a chemical sampling apparatus configured to collect, store and/or analyze samples. Atmospheric samples, for example, may be collected by the sampling device and include atmospheric gasses like oxygen and nitrogen, that contain materials to be analyzed, including potentially harmful chemical contaminants or pollutants, biological materials (e.g., anthrax spores), and radioisotopes. The training data may represent the response of one or more sampling devices that may include a FAIMS detector, a Photo Ionization Detector, a Metal Oxide Detector, or other detector to detect the presence of chemicals in the atmosphere. The trained models 60 may be configured to receive and analyze atmospheric samples for detection and classification of one or more desired chemicals. The materials collected by the sampling device may be referred to as analytes.

The model/dataset performance results (which may include, for example, chemical classification errors) are provided to a model analysis engine 70. The model analysis engine 70 may include classification training and optimization algorithms, statistical analysis algorithms for identifying features/attributes associated with a target analyte, optimization algorithms for simplifying the number of variables, model selection algorithms for comparing trained models, validating trained models and selecting features and training dataset parameters for each model, preprocessing algorithms such as preprocessing and normalization algorithms, and other algorithms consistent with the teachings of the present disclosure. In various embodiments, the model analysis engine 70 may incorporate and/or be based on a generalized machine learning platform such Scikit-learn and/or TensorFlow.

The model analysis engine 70 receives informative metrics compiled during the training process 58 and validation process or the trained models 60, and configuration parameters 64 that define a scope of use for the trained models 60 (e.g., user identification of an end-user sampling device, chemical targets and use cases). The model analysis engine 70 may then analyze the received data to modify the training dataset samples used for training the models by identifying samples to retain (e.g., samples that contribute to proper classification), drop from (e.g., samples that do not contributed to proper classification) and/or add to the training dataset 56. In one or more embodiments, the model analysis engine 70 receives the informative metrics and performance results, analyzes the available data in view of the configuration parameters 64, and updates the training dataset 56 to train a model with improved results.

In various embodiments, the model analysis engine 70 includes various tools including a feature analyzer 72, a dataset generator 74, and an assembler/interface 76. The feature analyzer 72 receives the informative metrics and performance results, extracts features for further processing, and analyzes the relative performance of one or more samples from the training dataset 56 that was used for training one or more models. Metrics may include, for example, extracted features, data indicating changes in neural network parameters, data from previous iterations, and other data captured during training. Analysis of extracted features from the training data 56 may include analysis of analyte features of chemicals of interest from acquired samples that uniquely identify the chemicals, such as peak height and peak location data. In some embodiments, the feature analyzer 72 ranks the features based on performance results and optimizes the features to be used in the next iteration.

In various embodiments, the feature analyzer 72 may extract informative metrics and/or performance results into various categories for further analysis, including compiling data based on different classification labels of the chemical samples from the training dataset, data based on performance/underperformance, sample characteristics (e.g., features extracted), and other groupings as may be appropriate. The feature analyzer 72 may analyze IMS spectra data to identify features such as edge of peaks, gaussian peak heights, peak locations, etc.

The dataset generator 74 analyzes the training samples from the training dataset 56 based on the performance results and/or the effect the sample had on the training of the model. The dataset generator 74 generates parameters for a new training dataset 56 that may include a subset of current training dataset 56 samples and parameters defining new training datasets to be generated for the next training dataset. The assembler/interface 76 provides a user interface to communicate user defined configuration parameters to the model analysis engine and provide feedback to the user on model analysis results, including data, rankings and options regarding features, data samples and models. In some embodiments, the process continues iteratively until the final training datasets and models 80 that meet certain performance criteria, such as a percentage of correctly classified chemical samples during the validation process, performance for various chemical types/sampling conditions, cost validation and/or other criteria, is generated. The trained models may then be used, for example, in end-user devices to detected and classify one or more chemicals.

In one or more embodiments, the dataset generator 74 includes one or more algorithms, neural networks, and/or other machine learning processes that receive the informative metrics and performance results and determines modifications of the training dataset to improve performance. The configuration parameters 64 define one or more goals of the classification models, such as parameters defining labels, chemicals, and environments to be used in the training dataset. For example, the configuration parameters 64 can be used to determine what chemicals the neural network should classify and environments in which the chemicals should appear.

In various embodiments, model analysis engine 70 and/or other components for generating the training dataset may include a synthetic sample generator that receives instructions/parameters to create new training samples. Synthetic sample generation may include construction of defined and/or random synthetic samples, informed by configuration parameters 64 and an identification of desirable and undesirable parameters as defined by the dataset generator 74. For example, the trained models 60 may be configured to label certain chemicals in a variety of real world environments, and the current training dataset may be producing unacceptable results classifying chemicals in certain of the environments. The synthetic sample generator may be instructed to create sample data of a certain chemical classification having a range of features in particular environments in accordance with the received parameters. For example, by modifying existing data samples to create new data samples representing a desired environment.

In some embodiments, the dataset generator 74 determines a subset of samples from the training dataset 56 to maintain in the training dataset and defines new samples to be selected and/or generated. In some embodiments, samples from the training dataset 56 may be ranked on performance results by ranking each sample's impact based on overall performance. For example, the dataset generator 74 may keep a number of top ranked samples for each chemical classification, keep samples that contribute above an identified performance threshold, and/or keep a certain number of top ranked samples overall. The dataset generator 74 may also remove samples from the training dataset 56 that are lowest ranked and/or contribute negatively or below an identified performance threshold.

In operation, the system 50 iteratively trains and validates each model and produces performance data (e.g., a table of results) that identifies the relatively accuracy of each model. The performance data may include an identification of the useful features and contributions of the training data samples to the various models. For example, a user may select a set of models and features to test, and the performance data provides a list of tested models, the accuracy of each model and the set of relevant features identified during the test. In some embodiments, the system 50 iteratively splits the dataset into training dataset and a validation dataset, for example, by randomly selecting data for the training and validation datasets. In some embodiments, the accuracy of a feature, contribution of a data element or contribution of other parameters may be determined by running the models under various scenarios that include, exclude or modify the tested feature, data element and/or parameter, and comparing the performance results. After running various scenarios, the importance of each feature, data element and parameter can be determined and used to optimize the models in a next iteration of the process.

An example operation of the system 50 for training, validating and selecting one or more models for classifying a chemical will now be described in further detail with reference to FIG. 1B. A training and validation method 100 includes a data collection step 110 in which chemical samples are collected and processed to create training and validation datasets for one or more chemical targets. In various embodiment, the chemical data can include real-world samples collected by a chemical sensor in the field in various environments, chemical data collected in controlled laboratory environments, and/or synthetic/modified chemical data generated to produce a more robust training dataset. In various embodiments, the data is collected in the form of IMS spectra data, such as represented by the graph 190 in FIG. 1C. Generally, IMS spectra represents the intensity of ion peaks versus the drift time of ions through an ion mobility spectrometer. FAIMS, for example, separates ions at atmospheric pressure based on the difference between the mobility of an ion in strong and weak electric fields. The field-dependent mobility of an ion is measured with respect to the compensation voltage (V) at which the ion is transmitted through the FAIMS at an applied asymmetric waveform dispersion voltage. Each analyte has a unique two-dimensional spectrum that is used to classify the analyte.

After collection of the data, the training dataset is constructed and verified (step 120) and pre-processing steps 130 are performed (e.g., feature extraction) to generate input data for one or more machine learning models. The training data and features are processed and refined using peak fitting and/or other statistical approaches to measure and optimize performance (step 140). For example, in some embodiments, the mean zero air is subtracted from the chemical data to calculate a mean for an analyte. A Gaussian peak fitting and refinement process smooths the chemical spectra. In some embodiments, peak selection algorithms, peak fitting algorithms, cluster analysis algorithms and/or other statistical algorithms made be used to identify a set of features. The process analyzes the relative importance of each feature and further iterations can be performed to optimize the feature set (e.g., identify additional features and/or select a subset of features) and feature parameters to refine the model. In some embodiments, a feature set is selected that will work across a range of target chemicals for a particular implementation.

Next, the models are optimized including pre-processing in step 150 (e.g., feature extraction), determination of best fit/performance (step 160) and verification of the trained models (step 170). Features may include, for example, compensation values of peaks having values representative of the height of each peak. In some embodiments, the features may be normalized to accommodate data having different ranges/values. The data input to the model may include an array of n observations/samples (rows) and m features/compensation values (columns) and a vector ray of n labels. The labels may represent the chemical compound observed in each sample. In various embodiments, model types may include decision trees, support vector machines, logistic regression, k-Nearest-Neighbors, Naïve Bayes classifiers, and other model types. The models are fit to the training dataset, which may include 10,000 or more labeled training samples. In some embodiments, the training dataset is randomized for use in the model training process. In various embodiments, the training dataset is adapted to minimize under-fitting and over-fitting. The models are validated using a separate validation dataset and various analytics are produced, for example, an estimation of the relative importance of each feature (e.g., relative importance of various compensation voltages to the model).

In some embodiments, a model is trained for multi-class classification of multiple analytes, for example, dimethyl methylphosphonate, 2-chloroethyl ethyl sulfide, methyl salicylate and amyl acetate. The training could include, for example, samples of each analyte from multiple instruments under multiple environmental conditions, to generate a trained model configured to predict a probability of each classification for an input data sample. The trained model can then be implemented in a mobile chemical detection system for target detection. The trained machine learning models can provide improved classification that reduce minimum alarm levels and increase probability of detection, more efficiency in classifier development and more flexibility with expanding threat libraries.

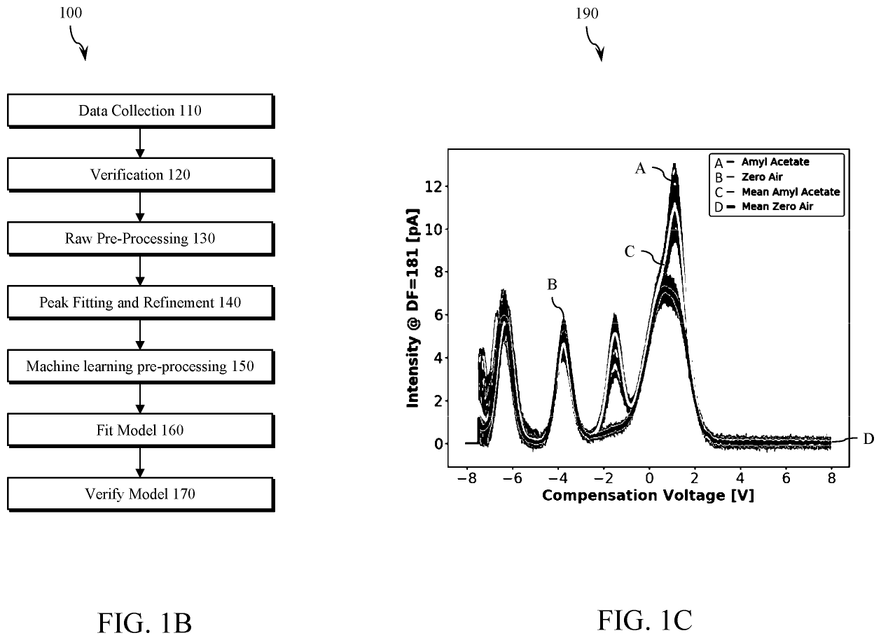

Referring to FIG. 2, various embodiments of a chemical classification system 200 will be described. The chemical classification system 200 may be implemented on one or more servers such as an application server that performs data processing and/or other software execution operations for generating, storing, classifying and retrieving data samples. In some embodiments, the components of the chemical classification system 200 may be distributed across a communications network, such as network 222. The communications network 222 may include one or more local networks such as a wireless local area network (WLAN), wide area networks such as the Internet, and other wired or wireless communications paths suitable for facilitating communications between components as described herein. The chemical classification system 200 includes communications components 214 operable to facilitate communications with one or more network devices 220 over the communications network 222.

In various embodiments, the chemical classification system 200 may operate as a general-purpose chemical classification system, such as a cloud-based system providing classification to a plurality of network devices (e.g., network device 220), or may be configured to operate in a dedicated system that identifies and classifies samples using a database 202. The chemical classification system 200 may be configured to receive one or more chemical data samples from one or more network devices 220 and process associated chemical identification/classification requests.

As illustrated, the chemical classification system 200 includes one or more processors 204 that perform data processing and/or other software execution operations for the chemical classification system 200. The processor 204 may include logic devices, microcontrollers, processors, application specific integrated circuits (ASICs), or other devices that may be used by the chemical classification system 200 to execute appropriate instructions, such as software instructions stored in memory 206 including dataset generation component 208, model training and analysis component 210, and trained chemical classification models 212 (e.g., a neural network trained by the training dataset), and/or other applications. The memory 206 may be implemented in one or more memory devices (e.g., memory components) that store executable instructions, data and information, including image data, video data, audio data, network information. The memory devices may include various types of memory for information storage including volatile and non-volatile memory devices, such as RAM (Random Access Memory), ROM (Read-Only Memory), EEPROM (Electrically-Erasable Read-Only Memory), flash memory, a disk drive, and other types of memory described herein.

Each network device 220 may be implemented as a computing device such as a portable chemical sampling device, computer or network server, a mobile computing device such as a mobile phone, tablet, laptop computer or other computing device having communications circuitry (e.g., wireless communications circuitry or wired communications circuitry) for connecting with other devices in chemical classification system 200.

The communications components 214 may include circuitry for communicating with other devices using various communications protocols. In various embodiments, communications components 214 may be configured to communicate over a wired communication link (e.g., through a network router, switch, hub, or other network devices) for wired communication purposes. For example, a wired link may be implemented with a power-line cable, a coaxial cable, a fiber-optic cable, or other appropriate cables or wires that support corresponding wired network technologies. Communications components 214 may be further configured to interface with a wired network and/or device via a wired communication component such as an Ethernet interface, a power-line modem, a Digital Subscriber Line (DSL) modem, a Public Switched Telephone Network (PSTN) modem, a cable modem, and/or other appropriate components for wired communication. Proprietary wired communication protocols and interfaces may also be supported by communications components 214.

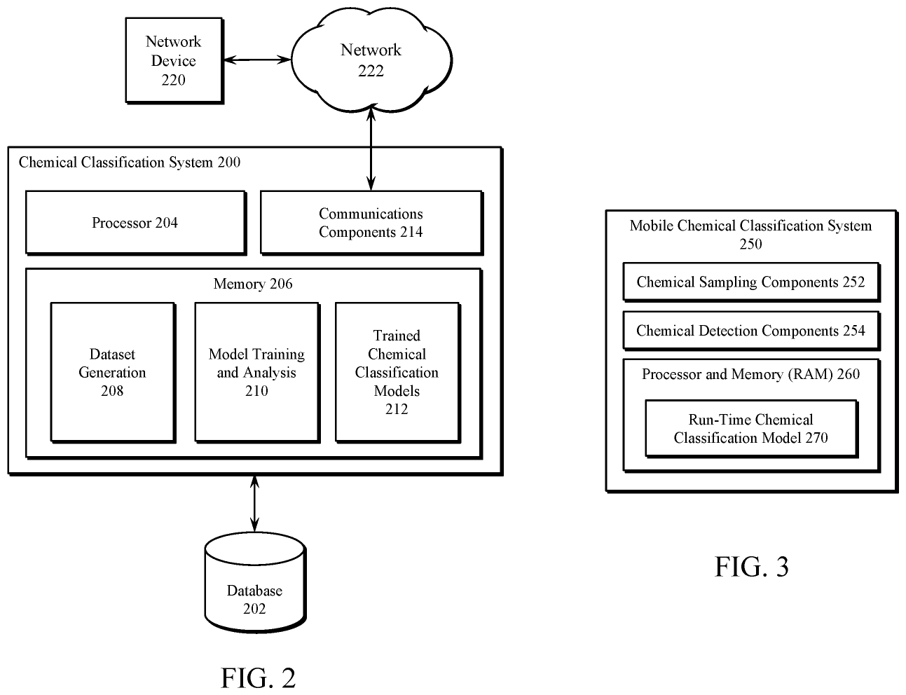

In various embodiments, a trained chemical classification system may be implemented in a real-time environment, as illustrated in FIG. 3. The mobile chemical classification system 250 may include a chemical sampling unit 252 and chemical detection components 254, configured to acquire and detect one or more chemicals of interest using, for example, a FAIMS sensor or other device or system configured to receive and/or generate ion mobility spectra data. In the illustrated embodiment, the mobile chemical classification system 250 includes a processor and memory 260, operable to store a trained chemical classification model 270 as described herein to classify the sampled analyte.

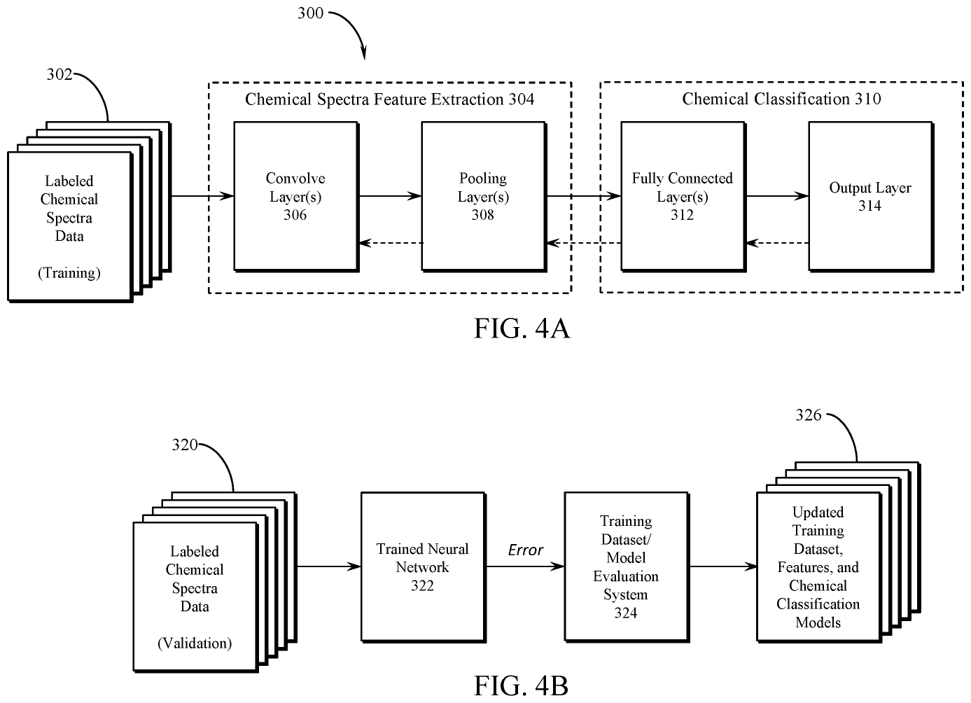

Referring to FIG. 4A, an embodiment of a chemical classification training process will now be described. In one embodiment, the chemical classification model 300 is a convolutional neural network (CNN) that receives labeled chemical spectra data from training dataset 302 and outputs a chemical classification. The training dataset may include chemical data acquired using a chemical sensor during various real-world conditions. In one embodiment, the training starts with a forward pass through the neural network including chemical spectra feature extraction 304 in a plurality of convolution layers 306 and pooling layers 308, followed by chemical classification 310 in a plurality of fully connected layers 312 and an output layer 314. Next, a backward pass through the neural network may be used to update the CNN parameters in view of errors produced in the forward pass (e.g., misclassified chemicals). In various embodiments, other neural network processes may be used in accordance with the present disclosure.

An embodiment for validating the trained chemical classification model is illustrated in FIG. 4B. A validation dataset 320 representing chemical sensor data is fed into the trained neural network 322. The validation dataset 320 represents a variety of chemicals, sensor readings and environments to classify chemicals. In some embodiments, a database of labeled chemical spectra data is constructed and a first subset of labeled chemical spectra data is used for training and a second subset of labeled chemical spectra data is used for the validation dataset. Detected errors (e.g., chemical misclassification) may be analyzed and fed back to the training dataset/model evaluation system 324 to optimize the training dataset, features, and trained chemical classification models 326. In various embodiments, detected errors may be corrected by adding more examples of sensor data for a chemical, removing sensor data from the training dataset, adjusting feature extraction criteria, and other adjustments to the training dataset and model generation process. In some embodiments, the system is configured to generate trained chemical classifications models representing a variety of scenarios, which are compared during the optimization processing.

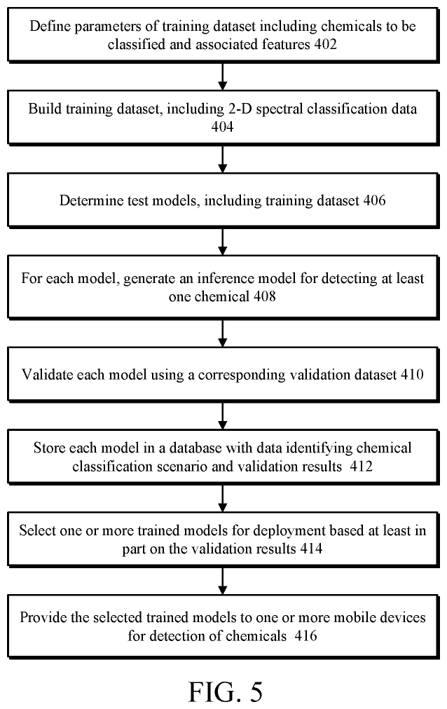

Referring to FIG. 5, embodiments of a process for generating training data for chemical detection will now be described. In step 402, an operator defines the parameters for the training dataset including an identification of the chemicals to be detected and classified, the chemical sensors and data to be modeled, associated features to be extracted from the data, and use cases/environments in which the samples will be captured. In step 404, a training dataset including 2D spectral classification data is defined to model the use case/environments. Next, modeling and dataset scenarios are determined, in step 406. For each model, an inference model is generated to detect at least one chemical, in step 408. In step 410, each model is validated using a corresponding validation dataset. In step 412, each model is stored in a database with data identifying the chemical classification scenario and validation results. In step 414, one or more trained models are selected for deployment based on the validation results, and in step 416, the selected trained models are downloaded or otherwise transferred to one or more mobile devices for chemical detection.

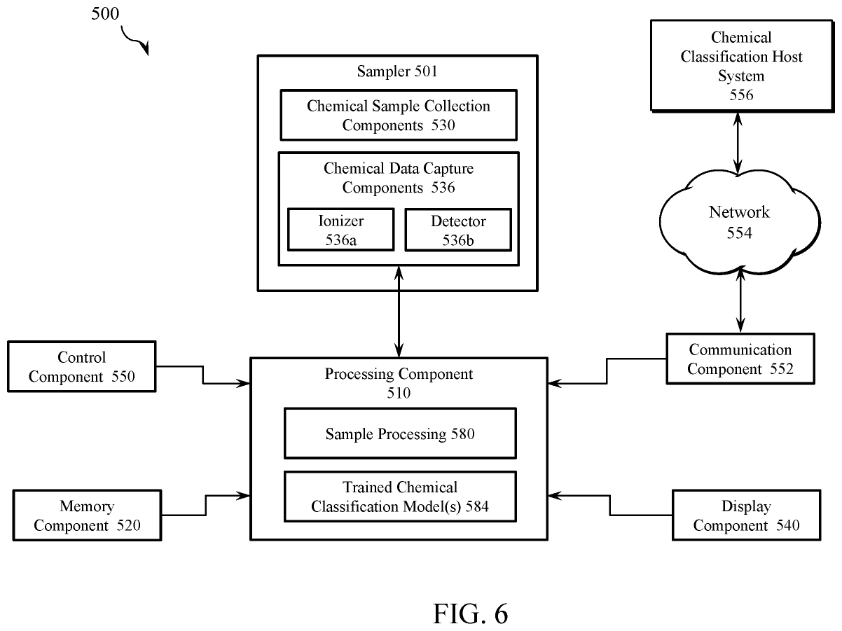

Referring to FIG. 6, various embodiments of systems for sampling and detecting chemicals will be described. A chemical classification system 500 may include a sampler 501 for collecting samples and capturing detected chemical data and processing components 510 for classifying one or more chemicals from the captured data. In one implementation, the sampler 501 and processing components 510 are embodied in mobile chemical sampler, such as a small, lightweight, battery operated device that can easily be transported to an area of possible contamination.

The sampler 501 may include chemical sample collection components 530 configured to acquire a sample for testing and chemical data capture components 536 configured to capture chemical data from a collected sample. The sampler 501 may include components to enable operators to analyze gas, liquid, or solid samples. In some embodiments, the chemical sample collection components 530 include one or more sampling components (e.g., syringe, cartridge, sample probe, etc.) that is used to sample the matter for analysis by the sampler 501. The sampler 501 may include an electronic interface, inlet and outlet ports, and/or other features as applicable for a particular implementation. The chemical sample and collection components 530 may further include a sample pump to pull air through the cartridge via an inlet and a flow/volume sensor to measure the sample volume, and a filter to filter debris and other solid or liquid particulates as desired. The chemical sample collection components 530 may have one or multiple sample flow paths to allow for sampling of sequential or simultaneous sampling of multiple samples. The intake system may be adapted to draw in, for example, gasses bearing solid or liquid particulates, liquids, or colloidal suspensions.

In some embodiments, the chemical sample collection components 530 include an aerosol or chemical agent detector, which may be a hand-held mobile device, platform-mounted mobile device or a standalone device in a laboratory. The chemical sample collection components 530 and chemical data capture components 536 may be configured for use with rapid thermal modulation ion spectrometry (RTMIS). RTMIS provides various advantages over IMS and FAIMS, including lower ion residence times and quicker scanning.

In various embodiments, the sampler 501 (and/or the processing component 510) may be configured to record information pertinent to the collected sample including GPS location when sampled, volume of sample collected, date/time stamp, voice data, and image data for use when the sample is analyzed. In some embodiments, the sampler 501 may include a FAIMS detector, a photo ionization detector, or a metal oxide detector to detect the presence of chemicals to alert the user to obtain a sample. The chemical data capture components 536 include components configured to perform a chemical analysis on the analytes in the sample. The chemical data capture components 536 may be any instrument for performing chemical analysis and generating a spectra data as described herein. In some embodiments, the chemical data capture components 536 may include a chemical separation device, such as, e.g., a gas-chromatograph (GC), a combination GC/MS, GC/electron capture detector (ECD), GC/FID, or other device. For example, chemical data capture components may include a gas chromatograph that separates the sample into individual targets and an ion mobility spectrometer analyzes each target to produce sample spectra for further analysis. The ion mobility spectrometer may operate, for example, by separating ions in an electric field based on their mobilities in a carrier buffer gas (e.g., using components such as an ionizer 536a) and driving the separated ions to a detector 536b through a drift tube. The detector 536b measures the separated ions in order of arrival, and the resulting chemical spectra provides a chemical fingerprint for the underlying target.

The chemical spectra data is provided to the processing component 510 for further analysis. In various configurations, the chemical classification system 500 may be configured to detect threats such as explosives and chemical and biological warfare agents, illegal drugs or other chemicals of interest. Example targets may include trinitrotoluene (TNT), C-4, pentaerythritol tetranitrate (PETN), RDX, ethylene glycol dinitrate (EGDN), hexamethylene triperoxide diamine (HMTD), triacetone triperoxide (TATP), urea nitrate, ammonium nitrate and other chemicals.

The processing component 510 may include, for example, a microprocessor, a single-core processor, a multi-core processor, a microcontroller, a logic device (e.g., a programmable logic device configured to perform processing operations), a digital signal processing (DSP) device, one or more memories for storing executable instructions (e.g., software, firmware, or other instructions), and/or any other appropriate combination of processing device and/or memory to execute instructions to perform any of the various operations described herein. Processing component 510 is adapted to interface and communicate with components the sampler 501 and components 520, 540, 550 and 552 to perform method and processing steps as described herein. Processing component 510 is also adapted to detect and classify chemicals in the chemical data captured by the sampler 501 through sample processing module 580 and one or more trained chemical classification modules 584.

It should be appreciated that processing operations and/or instructions may be integrated in software and/or hardware as part of processing component 510, or code (e.g., software or configuration data) which may be stored in memory component 520. Embodiments of processing operations and/or instructions disclosed herein may be stored by a machine-readable medium in a non-transitory manner (e.g., a memory, a hard drive, or a flash memory) to be executed by a computer (e.g., logic or processor-based system) to perform various methods disclosed herein.

Memory component 520 includes, in one embodiment, one or more memory devices (e.g., one or more memories) to store data and information. The one or more memory devices may include various types of memory including volatile and non-volatile memory devices, such as RAM (Random Access Memory), ROM (Read-Only Memory), EEPROM (Electrically-Erasable Read-Only Memory), flash memory, or other types of memory. In one embodiment, processing component 510 is adapted to execute software stored in memory component 520 and/or a machine-readable medium to perform various methods, processes, and operations in a manner as described herein. Processing component 510 may be adapted to receive chemical data from the sampler 501, process and/or store the chemical data, and/or retrieve stored chemical data from memory component 520. Processing component 510 may further be adapted to classify one or more chemicals using trained chemical classification models 584 as described herein.

Display component 540 may include an image display device (e.g., a liquid crystal display (LCD)) or various other types of generally known video displays or monitors. The display component 540 may be used to display information related to operation of the sampler 501 as well as other information about the sample, sample cartridge, or the environment.

Control component 550 may include, in various embodiments, a user input and/or interface device, such as a keyboard, a control panel unit, a graphical user interface, or other user input/output. Control component 550 may be adapted to be integrated as part of display component 540 to operate as both a user input device and a display device, such as, for example, a touch screen device adapted to receive input signals from a user touching different parts of the display screen. In one or more embodiments, the control component 550 may be used to select an operation mode or to enter data about the sample or sample cartridge. Different operation modes may be selected that operate the apparatus according to varying parameters. For example, an operation mode may be selected that operates a sample pump for a predetermined length of time. Another operation mode may be selected that operates a sample pump until a predetermined volume of gas has passed through a flow meter. Various operation modes may be programmed into the memory of the processing component 510 by a user, as unique operation modes are developed.

Communication component 552 may be implemented as a network interface component adapted for communication with a network including other devices in the network and may include one or more wired or wireless communication components. In various embodiments, a network 554 may be implemented as a single network or a combination of multiple networks, and may include a wired or wireless network, including a wireless local area network, a wide area network, the Internet, a cloud network service, and/or other appropriate types of communication networks.

In various embodiments, chemical classification system 500 provides a capability, in real time, to detect and classify chemicals in a sample. Chemical data from a sample may be received from the sampler 501 by processing component 510 and stored in memory component 520. The sample processing module 580 may process the chemical data for use by the trained chemical classification modules 584, for transmission to a remote device (e.g., chemical classification host system 556) or for other uses depending on the configuration of the chemical classification system 500. The trained chemical classification module 584 detects and classifies one or more chemicals in the sample data and stores the result in the memory component 520, an object database or other memory storage in accordance with system preferences. In some embodiments, chemical classification system 500 may send sample data or classification results over network 554 (e.g., the Internet or the cloud) to a server system, such as chemical classification host system 556 for further processing. In some embodiment, the processing components 510 are configured to trigger a notification or alarm to the user (e.g., through the control component 550 or display component 540) when a chemical of interest is detected in the environment and should be sampled.

Where applicable, various embodiments provided by the present disclosure can be implemented using hardware, software, or combinations of hardware and software. Also, where applicable, the various hardware components and/or software components set forth herein can be combined into composite components comprising software, hardware, and/or both without departing from the spirit of the present disclosure. Where applicable, the various hardware components and/or software components set forth herein can be separated into sub-components comprising software, hardware, or both without departing from the spirit of the present disclosure.

Software in accordance with the present disclosure, such as non-transitory instructions, program code, and/or data, can be stored on one or more non-transitory machine-readable mediums. It is also contemplated that software identified herein can be implemented using one or more general purpose or specific purpose computers and/or computer systems, networked and/or otherwise. Where applicable, the ordering of various steps described herein can be changed, combined into composite steps, and/or separated into sub-steps to provide features described herein.

Embodiments described above illustrate but do not limit the invention. It should also be understood that numerous modifications and variations are possible in accordance with the principles of the invention. Accordingly, the scope of the invention is defined only by the following claims.

What is claimed is:

1. A system comprising:
a storage device configured to store a training dataset of labeled chemical spectra data, chemical classification models, performance criteria and results; and
a logic device configured to optimize the training dataset and chemical classification models for chemical classification through a process comprising:
determining at least one subset of the labeled chemical spectra data for training one or more of the chemical classification models;
determining at least one feature set for extracting features from the labeled chemical spectra data;
training a plurality of chemical classification models to classify one or more chemicals using the subset of labeled chemical spectra data and the feature set, and generating associated training metrics;
validating the trained chemical classification models and generate performance results; and
analyzing each trained chemical classification model using the associated training metrics and performance results and updating the subset of labeled chemical data, the feature set, and/or the training chemical classification models to optimize performance.

2. The system of claim 1, wherein the system is further configured to generate informative metrics representing a contribution of sample from the chemical spectra data to the trained chemical classification models.

3. The system of claim 2, wherein the logic device is further configured to execute a training dataset analysis engine configured to generate new chemical spectra data training dataset in response to the informative metrics.

4. The system of claim 1, wherein the logic device is further configured to execute a model validation system comprising a validation dataset comprising a plurality of labeled, chemical sample data, wherein the trained chemical classification model classifies chemicals from the validation dataset.

5. The system of claim 4, wherein the logic device is further configured to collect, and store labeled chemical spectra data.

6. The system of claim 1, wherein the logic device further comprises a feature analyzer configured to receive informative metrics and evaluate features of the chemical spectra data based on a contribution to the chemical classification model.

7. The system of claim 1, wherein the logic device further comprises a dataset generator configured to define an updated training dataset comprising a subset of the training dataset and parameters for a chemical sample data to be generated.

8. The system of claim 7, wherein the logic device further comprises an assembler/interface configured to process model parameters and generate instructions to control the logic device to generate chemical classification models in accordance with the parameters.

9. The system of claim 8, wherein the model parameters define a scope of the chemical classification models including at least one chemical to classify.

10. The system of claim 1, wherein the logic device is further configured to rank each sample of the chemical spectra data on a relative contribution to a performance of the chemical classification model.

11. A method comprising:
storing a training dataset of labeled chemical spectra data, chemical classification models, performance criteria and results;
optimizing the training dataset and chemical classification models for chemical classification through processes comprising:
determining at least one subset of the labeled chemical spectra data for training one or more of the chemical classification models;
determining at least one feature set for extracting features from the labeled chemical spectra data;
training a plurality of chemical classification models to classify one or more chemicals using the subset of labeled chemical spectra data and the feature set, and generating associated training metrics;
validating the trained chemical classification models and generate performance results; and
analyzing each generated chemical classification model using the associated training metrics and performance results and updating the subset of labeled chemical data, the feature set, and/or the trained chemical classification models to optimize performance.

12. The method of claim 11, further comprising generating informative metrics representing a contribution of each sample from the chemical spectra data to the trained chemical classification models.

13. The method of claim 12, further comprising executing a training dataset analysis engine configured to generate new chemical spectra data training dataset in response to the informative metrics.

14. The method of claim 11, further comprising executing a chemical classification model validation system comprising a validation dataset comprising a plurality of labeled, chemical sample data, wherein the trained chemical classification model classifies chemicals from the validation dataset.

15. The method of claim 14, further comprising collecting and storing labeled chemical spectra data.

16. The method of claim 11, further comprising analyzing features by receiving informative metrics and evaluating features of the chemical spectra data based on a contribution to the chemical classification model.

17. The method of claim 11, further comprising generating a dataset by defining an updated training dataset comprising a subset of the training dataset and parameters for a chemical sample data to be generated.

18. The method of claim 17, further comprising executing an assembler/interface configured to process model parameters and generating chemical classification models in accordance with the parameters.

19. The method of claim 18, further comprising defining a scope of the chemical classification models including at least one chemical to classify.

20. The method of claim 11, further comprising ranking each of the plurality of chemical spectra data on a relative contribution to a performance of the chemical classification model
